(19) United States
(12) Patent Application Publication
(10) Pub. No.: US 2024/0017089 A1
(43) Pub. Date: Jan. 18, 2024

(54) System and method for treating the nails

(71) Applicant: EUROFEEDBACK, LISSES (FR)

(72) Inventor: Georges SAFRAOUI, VILLEJUST (FR)

(21) Appl. No.: 18/039,991

(22) PCT Filed: Nov. 29, 2021
(86) PCT No.: PCT/EP2021/083366

§ 371 Date: Jun. 2, 2023

(30) Foreign Application Priority Data
Dec. 3, 2020 (FR) 2012647

(51) Int. Cl. A61N 5/06; A45D 29/00; A45D 29/17

(52) U.S. Cl. CPC A61N 5/0625; A61N 5/062; A61N 5/0624; A45D 29/004; A45D 29/17; A61N 2005/0626; A45D 34/042

(57) ABSTRACT

A system for treating nails, comprising:
a pulse light emission device (1) making it possible to expose at least one nail to be treated to at least one light pulse, even better to a burst of light pulses,
at least one coating (16) to be applied to the nail or nails, absorbing the light emitted by the device, or an applicator of such a coating, notably of reflectivity less than 50% and/or transmissivity less than 50%, and the surface temperature of which can exceed 60° C., and more preferentially 200° C., under the effect of the light emitted by the device.

### TECHNICAL FIELD

The present invention relates to the treatment of nails of the hands and feet.

### PRIOR ART

Many people, above all elderly people, suffer from various affections of the nails, notably onychomycoses.

The conventional treatments which rely on the use of antifungal varnishes, are relatively restrictive because they have to be performed over a long period, and also have the drawbacks that are inherent in the use of compounds that exhibit a certain toxicity.

The application US 2013/211481 describes a coating of hardening liquid dressing type, infused with an antifungal agent, that can include a colorant which changes color at a certain temperature when exposed to the light source.

It is known practice to use light, in particular pulsed light, to try to improve the esthetic appearance of the nail or its pathological condition.

The U.S. Pat. No. 6,090,788 discloses a phototherapy method for treating the fungal infections of the nail, in which the pathogen agent has associated a pigment that absorbs light before its irradiation by a light beam.

The applications US 2009/143842, US 2014/288621, US 2014/194955 and US 2012/109265 disclose other light devices for treating fungal or bacterial nail infections.

Other trials of treatments by pulsed light have been carried out to try to improve the esthetic appearance of the nail or its pathological condition. The preparation of the nails for the treatment is then limited to a possible abrading of the outer surface of the nail in order to remove the overthicknesses. The nail is then exposed for a few minutes to a sequence of repeated flashes, of sufficient fluence to raise the temperature of the nail.

In the case of a polychromatic flash lamp (IPL), the divergent light flashes are sent from a distance that is sufficient not to burn the cutaneous environment of the nail, and the temperature at the surface of the nail is situated below 50° C. (45° C. being considered the limit of the zone of discomfort). In general, 4 to 6 sessions at 3- to 4-week intervals are performed.

In the case of the use of a laser light source, it is the YAG solution at 1054 nm which is used. The zone treated by the laser is limited to a spot of a few mm², which requires numerous firings to treat the entire surface of the nail. Furthermore, the fluence is relatively great, of the order of 70 J/cm², because of the low absorption of the tissues. As for the treatment by polychromatic flash lamp, the temperature does not exceed 50° C. on the surface of the nail in order not to generate burns or discomfort.

The effectiveness of such treatments is not demonstrated, because of the low temperature reached. Furthermore, the duration of the laser treatment is relatively lengthy, of the order of several minutes per nail, which renders the treatment tedious.

Thus, the treatments of the nails by light, as performed currently, remain relatively ineffective, as testified by various studies such as that which is the subject of the article entitled “The effectiveness of lasers in the treatment of onychomycosis: a systematic review” Bristow Journal of Foot and Ankle Research 2014, 7:34.

### DISCLOSURE OF THE INVENTION

There consequently remains a need to improve the effectiveness of the treatments of the nails by light with a view to improving their esthetic appearance and/or treating pathologies such as onychomycoses.

### SUMMARY OF THE INVENTION

The invention aims to address this need, and it achieves this according to a first of its aspects by virtue of a method for preparing a nail in order for it to undergo a treatment by light pulses, this method comprising the step of applying to the nail a coating absorbing the light emitted during the treatment, notably a coating of dark color, in particular black, and in positioning an output window of a light pulse emission device relative to the nail such that the light emitted by the device is directed toward the nail.

“Absorbent coating” should be understood to mean that the coating absorbs a significant proportion of the light emitted by the light pulse emission device, and improves the transformation of the light into heat compared to the bare nail. Advantageously, the reflectivity R of the coating is less than 50%, better than 40%, 30% or 20%, and/or its transmissivity T is less than 50%, better 40%, 30% or 20%.

Preferably, there is a reflectivity and/or a transmissivity of the coating according to the invention that are less than 50%, better than 40%, 30% or 20%, compared to a so-called SFL (Super Filtered Light) spectrum light that can be used for the treatment, the spectral distribution of the intensity of which is represented in FIG. 13. It can be seen in this figure that the substantial intensity is emitted after approximately 675 nm (in the solid part of the figure), the emission spectrum extending to around 1200 nm. A light line represents the spectrum emitted below 675 nm by the lamp, a filter internal to the device blocking the outgoing light to give the emission spectrum corresponding to the solid part. The SFL spectrum is for example emitted by a machine of FLUENCE, ARIANE or ANTHELIA brand from the company EUROFEEDBACK.

The reflectivity R corresponds to the portion of light energy reflected Er at the surface of the coating, added to the incident energy Eo, given the emission spectrum thereof. It varies from 0 for black bodies to 1 for a perfect mirror.

The transmissivity T is defined as being the ratio of the energy of the transmitted light Et added to the energy of the incident light Eo. For a given reflectivity R, the less transmissive a body is, the more it is absorbent, with 1=R+T+A, in which A is the absorptivity.

Preferably, the absorptivity A of the absorbent coating (expressed as %) is greater than or equal to 50%, better 60%, even better 70%, even 80% or 90%, ideally more than 95% or 99%.

The transmissivity T can be measured with a precision joule-meter, by measuring the ratio between the energy of the incident light and that of the light outgoing through the coating.

For an example of coating made of black polyimide (Kapton), there is for example a transmissivity T less than 20%, for example of the order of 15%, and for another example of coating, made of black PTFE, there is a transmissivity less than 10%, for example of the order of 5%. In the first case, for an incoming energy of 30 J, there is approximately 4 J at the output, and in the second case, for an incoming energy of 30 J, there is approximately 2 J at the output.

For comparison, the natural reflectivity of the healthy nails is of the order of 50% (for a wavelength of 800 nm) for a nail thickness of 1 mm; the bare nails absorb, for example, only approximately 8% of the incident light, such that the energy absorbed by the nails is limited to approximately 4% of the incident energy.

The coating disposed on the nail makes it possible to artificially absorb the light emitted by the device and its temperature can increase very rapidly and be propagated in the form of a “thermal wave” through the nail to the underlying tissue. The temperature rise which results therefrom makes it possible to effectively destroy the mycoses or other pathogen agents which affect the nail, but without causing burning because of its short duration. Furthermore, since the opaque coating extends only over the nail, the cutaneous environment around the nail does not undergo the same temperature rise, and can be preserved. The invention makes it possible to enhance the effectiveness of the treatment of the nails with lesser power and fluence.

The invention allows a simultaneous treatment of several nails at the same time, if so desired, because the lower light power required can make it possible to better spread out the light.

The invention allows a selective and safe treatment, because it does not heat the skin beyond measure around the nail, and makes it possible to work on black skins, if necessary, by protecting the skin, as explained later.

The temperature on the surface of the coating is for example, at the end of the burst of pulses received, between 180 and 250° C., for a fraction of a second. For example, the temperature at the surface of the coating is of the order of 200° C. or more for approximately 0.2 s after the end of the burst of flashes. Despite this high temperature, which drops back rapidly at the end of the last flash, the user feels only a sensation of discomfort or a slight sensation of burning, but which disappears rapidly. The temperature under the nail reaches, for example, 70° C. for approximately 1 s, because the nail is a thermal insulator and also has a thermal time constant TRT (thermal relaxation time) of more than a second. All the molds having colonized the material of the nail and being fed thereby are for example subjected to temperatures ranging from 100° C. to 200° C. for a fraction of a second, but sufficient time to destroy them.

The powers can be chosen so as to treat only the keratinous part of the nail, and not the underlying tissues, at high temperatures, for example of the order of 180° C. on the surface and 100° C. at the center of the nail, which makes it possible to effectively and relatively rapidly to destroy the fungi while limiting the discomfort to the user. Thus, the keratinous part can be treated at a temperature greater than 65° C. while the underlying tissue part, vascularized and innovated, is not subjected to a temperature greater than 65° C.

The coating makes it possible to transform into heat a large fraction of the energy of the incident light, for example more than 20 times the energy which would be absorbed by the nail in the absence of coating.

Preferably, a reflective coating is applied to the periphery of the nail, notably white, preferably a water-dispersible coating. Such a coating makes it possible to reflect the light and thus even further limit the rise in temperature of the skin at the periphery of the nail. That makes it possible to subject the absorbent coating disposed on the nail to a burst of light pulses without fear of burning the skin extending in immediate proximity to the nail.

Preferably, the opaque coating consists of a preformed film, preferably a film resistant to a temperature greater than or equal to 60° C., better 80° C., even better 100° C., 150° C. or 200° C., even better 250° C., better 300° C., preferably made of polyimide or of PTFE. The temperature the coating withstands will be chosen as a function of the surface power on the coating; a resistance to the higher temperature makes it possible to achieve a higher temperature, which tends to improve the effectiveness of the treatment and to shorten it.

“Resistant to a temperature T” should be understood to mean that the coating does not decompose under the effect of the heat at that temperature T, notably does not burn, and that it is sold as being usable at that temperature.

“Preformed” should be understood to mean that the film is already cohesive before being applied to the nail. The opaque coating can be a film, preferably self-adhesive, resistant to temperature, preferably resistant to a temperature of at least 60° C., better of at least 80° C., even better of at least 100° C., preferentially of at least 150° C., and very preferentially of at least 200° C., better of at least 250° C., even better of at least 300° C., for example a film of black adhesive Kapton. This film can be present initially on a non-adhesive support sheet. The thickness of the film is preferably greater than or equal to 50 microns, better 100 microns. The thickness of the film is for example less than 1 mm, being preferably between 100 and 150 microns. Preferably, use is made of plastic materials of class H or above (according to the IEC 60085 standard concerning the thermal class of insulators) that can sustain for example 327° C. for PTFE and 400° C. for Kapton. Use can also be made of certain polyamides resistant to heat, among other usable materials.

The absorbent nature of the coating may be due to the presence of black or dark pigments or colorants, for example mineral pigments. The coating can comprise graphite or carbon nanotubes, the invention not being limited to a particular coating.

Preferably, the film is precut substantially in the format of the nail to be treated, or to the format of a part of the nail, thus forming a patch in one or more parts to be applied to the nail. It is notably possible to position on the nail at least two precut parts, each having a rounded edge that can be adjusted when being stuck onto the nail to best follow the form of the edge thereof, the two patches overlapping one another on the nail. This overlap, for example at the middle of the nail, allows the edges of the nails to be well covered. The patches can be cut manually or using any appropriate instrument or device.

Although the use of a preformed film constitutes a particularly rapid and effective means for preparing the nail, it is also possible, without departing from the scope of the invention, to apply the coating in the fluid state to the nail, using an applicator. For example, the coating is formed by the drying of a composition applied in the fluid state to the nail, for example an absorbent varnish resistant to temperature and comprising a dispersion of an opaque pigment in a binder, the pigment being preferably of dark color, notably black, for example a metal oxide, notably of iron or of carbon. This varnish can be film-forming, in order to be able to be peeled off. It can also be water-dispersible, in order to facilitate its removal. The binder of the varnish is preferably chosen to exhibit the necessary temperature resistance, notably resist a temperature greater than or equal to 60° C., better 80° C., even better 100° C., better 150° C., preferably 200° C., better 250° C., even better 250° C.

It is also possible to apply to the nail an opaque coating in the form of an ink or a powder, for example using a felt pen with absorbent ink, preferably of dark color, even better black, for example a black indelible ink. One drawback with the use of such ink can be the need to use a solvent for its removal, such that the use of a coating in the form of a preformed film is preferred.

The absorbent coating can, if appropriate, be deposited on a varnish previously applied, in order to be easily removable by peeling or cleaning. This other varnish can be transparent. This other varnish is resistant to temperature and preferably supports a temperature of 60° C., better 80° C., even better 100° C., better 150° C., preferentially 200° C. and more preferentially 250° C.

Preferably, the method for preparing the nail comprises the step of abrading the surface of the nail to be treated prior to the application of the absorbent coating. That makes it possible to smooth the surface of the nail and to reduce the thickness of the nail. The smoothing makes it possible to enhance the quality of the thermal contact of the coating with the nail, notably when the coating is in the form of a preformed film. The sanding of the nail can be performed so as to have a remaining nail thickness of between 1 and 2 mm, better approximately 1 mm, over substantially the entire surface of the nail, which will be covered by the absorbent coating.

Notably in the case where the light emitted is generated by at least one flash lamp, the nail is preferably positioned at a certain distance from the light output window, in order to relatively uniformly illuminate all of the coating present on the nail. The method can thus comprise the step of positioning a support such that the nail is situated at a distance of between 1 and 6 cm, notably 2 and 5 cm, from the light output window. Preferably, in the case of a laser source, the light output window can be positioned and the divergence of the beam chosen such that each laser pulse irradiates the entire surface of the absorbent coating present on the nail. The output window can be separated by a distance of 0 to 20 cm, for example, from the surface of the coating.

Preferably, the entire surface of the skin exposed to the light is protected as best as it can be, and thus, a protective screen, notably a sheet of a flexible and reflective material, in particular a sheet of paper or of a white fabric, can be disposed above the skin of the digit. There may remain zones of skin that are not protected from the light, but preferably that are not situated in the immediate vicinity of the nail. For dark skins, it is preferable to totally mask the skin.

When only one nail is to be treated, the digit bearing this nail can be isolated from the other digits by a protective screen, notably a sheet of a flexible and reflective material, in particular a sheet of paper or of a white fabric.

When preparing several nails to be treated simultaneously, notably two or three, even all those of the foot, the output window can be positioned so as to be able to simultaneously expose these nails to the light emitted.

Interdigital separators can be disposed between the digits, if necessary.

Once the treatment has been performed, the absorbent coating can be removed, for example by simple peeling in the case of an adhesive or electrostatically adherent preformed film.

Another subject of the invention, according to another of its aspects, is a method for improving the appearance of at least one nail, comprising the step of preparing the nail by the implementation of the method according to the invention as defined above, and subjecting the duly coated nail to at least one pulse, and better at least one burst of light pulses.

Each pulse can be of a fluence, measured on the coating, of between 0.1 and 10 J/cm², better between 0.5 and 5 J/cm², even better between 0.5 and 2 J/cm², for example of the order of 1 J/cm². The coating on the nail is for example subjected to an energy per cm² of between 5 and 30 J for the total duration of the burst, this duration being for example between 1 and 10 s, better between 2 and 10 s, for example between 3 and 6 s.

To obtain 1 J/cm² in the case of an IPL generating a divergent polychromatic light, the fluence at the output of the head of the IPL can be of the order of four times greater, for example of the order of 4 J/cm². Because of the distance between the head of the IPL and the coating and the divergence of the beam, the fluence measured on the coating is lesser, for example approximately of four times lesser.

The surface power density, measured on the coating, is for example on average during the burst between 0.5 and 10 W/cm², better between 1 and 10 W/cm², even better between 1 and 5 W/cm². For example, at 3 Hz, three flashes of 4 J/cm² each are emitted per second at the output of the device, which corresponding to 12 J/cm² per second, but, if, given the distance from the nail, the latter receives for example approximately four times less thereof, i.e. 3 J/cm², there is then an average surface power density of 3 W/cm². Each flash of 4 J/cm² can have a duration of a few tens of milliseconds.

It is possible to treat only a single nail at a time. It is also possible to simultaneously expose at least two nails to the light pulse, as mentioned above.

Preferably, the nail is subjected to a number of light pulses of between 2 and 100, better between 2 and 50, notably between 5 and 20.

The frequency of emission of the light pulses can range from 1 to 10 Hz, preferably from 1 to 5 Hz, notably 2 to 4 Hz. There can be approximately 1/f in s between each flash, the duration of a flash being very short (of the order of 10 or so milliseconds). For a relatively rapid frequency, the temperature of the nail does not have time to decrease substantially between two successive flashes and thus tends to increase progressively during the flashes of the burst, to become maximal at the end of the last flash of the burst.

After the emission of a burst, the nail is preferably left to cool for a duration of at least 30 s, better of at least 1 mn, even better a duration of between 1 and 5 mn, before it is subjected to a new burst.

The nail may be subjected to only two successive bursts during a treatment session. Two treatment sessions can be spaced apart by at least one week. The nail can be subjected as necessary to a greater number of bursts.

The light can be emitted by at least one polychromatic flash lamp (IPL) or by a laser. The use of a flash lamp IPL treatment device is practical, because it makes it possible to easily treat several nails simultaneously.

During the treatment, the maximum temperature measured at the surface of the coating can exceed 60° C., better 80° C., even better 100° C., better 150° C., better 200° C., preferably lying between 150° C. and 300° C. A temperature greater than 150° C., better 180° C., even better 200° C., is reached preferably for a duration of at least 0.1 s, better for a duration of between 0.1 and 0.5 s, even better for a duration of between 0.1 and 0.3 s. A temperature greater than 150° C. is preferred, because it makes it possible to shorten the treatment while offering good effectiveness. A lower temperature, associated with the longer treatment time, can be retained when the power of the light source is lower, while being careful to remain below the burn threshold.

Another subject of the invention, according to another of its aspects, is a system for the treatment of nails, notably for the implementation of the treatment method as defined above, comprising:
a pulsed light emission device making it possible to expose at least one nail to be treated to at least one light pulse, better to at least a burst of light pulses,
at least one coating absorbent to the light emitted by the device, to be applied to the nail or nails, preferably a coating of reflectivity less than 50% and/or of transmissivity less than 50%, notably a coating of dark color, or an applicator of such a coating, and the surface temperature of which can exceed 60° C., better 80° C., even better 100° C. or 150° C., better 200° C., under the effect of the light emitted by the device.

Each light pulse can have all or some of the characteristics mentioned above.

The absorbent coating can have all or some of the characteristics already mentioned above. The coating can notably be of transmissivity of less than or equal to 20% and/or of absorptivity greater than or equal to 50%. The system thus can comprise an opaque film absorbing the light emitted by the device, preferably of dark color, notably black, resistant to a temperature of at least 60° C., better of at least 80° C., even better of at least 100° C. or 150° C., preferentially of at least 200° C., better of at least 250° C., even better 300° C., even 350° C. or 400° C., notably made of PTFE or of polyimide, in particular of Kapton.

This film is preferably covered on its inner face to be applied to the nail with an adhesive sensitive to pressure. As a variant, it does not include adhesive and adheres electrostatically.

The film is preferably precut to form a patch in the form of a nail or of a part of the nail, or a set of precut patches each in the form of a nail or of a part of the nail, notably with parts intended to overlap on the nail. The film can be precut to form at least one patch having a [symbol] -shaped outline for example.

It is thus possible to have, on a support, notably antiadhesive, a set of patches that are notably self-adhesive or electrostatically adherent to the nail, resistant to heat, each D-shaped, grouped in pairs on the support, two patches of one pair being intended to be placed on a same nail with an overlap between them.

If necessary, the system can comprise an applicator to apply the coating in the fluid state to the nail, for example an applicator of brush or felt tip type.

The system can comprise an applicator of a reflective coating, notably white, to form a reflective screen on the skin at the periphery of the nail.

The system can comprise a device for mechanically abrading the surface of the nail, notably a nail sander or pedicure sander.

The device can have emission characteristics such that the light makes it possible to raise the temperature of the absorbent coating present on the nail during the burst and/or at the end thereof to a temperature greater than 60° C., better 80° C., even better 100° C., preferentially 150° C., better 180° C., even better 200° C.

Preferably, the pulsed light emission device makes it possible to emit a burst of light pulses at a frequency of between 0.5 and 20 Hz, better between 1 and 5 Hz, each pulse having a fluence such that, given the distance separating an output window of the coating device during the use, it is between 0.5 and 5 J/cm² on the surface of the coating, better 0.5 and 2 J/cm², the number of pulses being preferably between 2 and 20, better between 5 and 20, notably between 10 and 20.

Advantageously, the emission device emits substantially between wavelengths of 650 nm and 1200 nm, with, for example, a peak around 875 nm, the system comprising a pair of protective goggles and a protective mask for the operator, comprising a filter absorbing the light emitted by the emission device, this filter having a transmission factor less than or equal to 1% above approximately 650 nm. Preferably, the absorbent filter of the goggles is of blue color.

That allows the operator using the device not to be dazzled by the light emitted, and thus be able to thus easily control the application of the light during the implementation of the treatment.

The system advantageously comprises a support for maintaining an output window, by which the light leaves the device, at a predefined distance from a reception surface of the nail to be treated. The nail can be positioned on this reception surface, under the light output window.

This distance is for example between 1 and 6 cm. The surface of the absorbent coating can thus be located at a distance of 0 and 5 cm from the output window, for example. The distance can be chosen as a function of the divergence of the light, to entirely cover a nail, even two nails, even more. In the case of a laser, the divergence of which is adjustable, it is possible to have an output window further away, if necessary.

The support can comprise a soleplate defining the abovementioned reception surface, and a raised part under which the foot can be fitted, provided with at least one aperture under which at least one of the nails can be disposed, the optical head being disposed in or above the aperture. In an exemplary implementation, the optical head has an optical conduit, the dimension of which allows it to be fitted in the aperture, the optical head otherwise remaining in abutment on the support. The aperture can be reniform, and extend over all of the nails of the foot, such that the operator can displace the conduit in the aperture during the treatment to treat several nails in succession.

The support can comprise at least one interdigital separator disposed so as to be fitted between two digits when the nail to be treated is disposed under the output window. Such a separator can assist in correctly positioning the nails with respect to the light output window. The pulsed light emission device can comprise at least one flash lamp or one laser.

The system according to the invention can also comprise, as a variant or in combination with the support, a tubular end-fitting having an internal surface that is at least partially reflective, the end-fitting being fixed at one of its ends to the output window of the optical head of the device and comprising, at its other end, a zone of reception of at least one digit, such that the nail of this digit is positioned to receive the light emitted by the optical head.

Such an end-fitting makes it possible to position the optical head with good reliability with respect to the nail to be treated. Furthermore, the tubular form of the end-fitting and the reflective surface make it possible to guide the light to the nail to be treated, which can improve the efficiency of the treatment and protect the person to be treated from the light.

Preferably, the end-fitting is made of metal, in particular of aluminum, which allows it to be disinfected easily between two treatments.

The end-fitting can be positioned so as to rest on the digit or digits receiving the treatment. The reception zone can comprise at least one concave incurved edge coming into contact with the digit or digits, which makes it possible to maintain the end-fitting in position while protecting the skin at the periphery of the nail or nails to be treated from the light emitted.

The emission device can comprise a control panel making it possible to set the characteristics of the light emitted as a function of the pathology and/or of at least one characteristic of the nail, such as its thickness, this control panel making it possible, for example, to select a “nail treatment” preset when other applications are possible (for example depilation). For a thicker nail, the surface power will be able to be increased and/or there can be a greater number of flashes.

Another subject of the invention, according to another of its aspects, is a device for treating nails by the emission of light pulses, comprising:
a light output window,
a support for maintaining a predefined distance between the output window and a reception surface of the digit bearing the nail to be treated.

This distance can be adjustable, if necessary, so as to adjust, for example, the size of the digit to be treated (big toe or other toe for example). This distance can be such that the separation between the output window and the coating is between 0 and 6 cm, for example between 2 and 6 cm.

The support can be made of metal or any other material, for example a thermoplastic material. The support can be produced, if necessary, in a transparent thermoplastic material, or include at least a transparent part, for example making it possible to monitor the correct positioning of the nail during the treatment.

The support can be produced with a reniform aperture, disposed so as to be superposed on all the nails of a foot, making it possible to slide an optical conduit of the treatment head inside it, this optical conduit defining the output window, for successively treating more nails of one and the same foot.

Another subject of the invention is a range of duly defined supports, of different sizes, allowing the operator to choose the support suited to the size of the foot to be treated.

Another subject of the invention is a support intended to allow the treatment of nails by a light pulse emission treatment device, comprising a part configured to receive an optical head of the device, the support forming a reception zone for receiving at least one digit such that the nail of this digit is positioned to receive the light emitted by an output window for the light emitted by the optical head, this positioning being preferably such that the output window is situated at a distance of between 1 and 6 cm, better between 3 and 6 cm, from a surface on which the digit is positioned. This support advantageously comprises an interdigital separator.

Another subject of the invention is a support intended to allow the treatment of nails by an optical head of a light pulse emission treatment device, notably for the implementation of the treatment method according to the invention, comprising a soleplate defining the reception surface, and a raised part under which the foot can be fitted, provided with at least one aperture under which at least one of the nails can be disposed, the optical head of the device being able to be disposed in or above the aperture so as to emit toward the nails. The aperture can be reniform, and extend over all the nails of the foot. The aperture can be of dimensions chosen to guide an optical conduit of the optical head, in a movement scanning the different nails situated under the aperture.

Another subject of the invention, according to another of its aspects, is an end-fitting intended to allow the treatment of nails by an optical head of a light pulse emission treatment device, notably for the implementation of the treatment method according to the invention, the end-fitting comprising:
a tubular body having a reflective inner surface, preferably metallic, notably of aluminum, and comprising a reception zone for receiving at least one digit such that the nail of this digit is positioned to receive the light emitted by the optical head, and
means for fixing the tubular body to the optical head of the device.

The fixing means can comprise a thin strap, for example adjustable, that attaches around the optical head, or any other suitable type of fixing, notably magnetic or snap-fitting fixing means, and the like.

Another subject of the invention, according to another of its aspects, is a set of patches to be positioned on nails to be treated using a light pulse emission device, these patches being preferably self-adhesive, precut in the form of a nail or of a part of a nail, and produced in an absorbent film within the meaning of the invention, preferably of dark color, better black, resistant to a temperature of at least 60° C., better of at least 80° C., better of at least 100° C. or 150° C., preferentially of at least 200° C., better of at least 300° C., preferably made of polyimide or of PTFE. The set of patches can comprise single-part patches or two-part patches, notably each D-shaped, as mentioned above, intended to overlap on the nail, of different sizes. All the patches can be disposed on a support, preferably antiadhesive treated, in a sufficient number to treat all of the nails of a hand or of a foot. The absorbent film preferably exhibits a reflectivity less than 50%, better 20%, and/or a transmissivity less than 50%, better 20%, with respect to the light emitted by the light pulse emission device. This film preferably appears opaque to the naked eye, when observed in the light of day.

Another subject of the invention is a nail preparation kit, comprising a set of patches as defined above and an applicator of a reflective coating, notably white, preferably water-dispersible.

Such a kit can further comprise a set of single-use abrasives, notably in the form of abrasive cylinders, arranged to be mounted on a sander to abrade the nail before the placement of the film.

The kit can further comprise the support according to the invention, as defined above.

The kit can also comprise a vacuum device for sucking up the dust created in the sanding of the nail.

The kit can also comprise a solution for cleaning the sanded nail before the application of the coating, notably an alcoholic aqueous solution.

### BRIEF DESCRIPTION OF THE DRAWINGS

The invention will be better understood on reading the following detailed description of nonlimiting exemplary implementations thereof, and on studying the attached drawing, in which:

FIG. 1 schematically represents an example of treatment device according to the invention,

FIG. 2 represents a schematic and partial cross-section of a nail provided with a coating according to the invention,

FIG. 3 illustrates the positioning of the nail under the light output window,

FIG. 4 is a block diagram illustrating different steps of the treatment method according to the invention,

FIG. 5 illustrates the sanding of the nail,

FIG. 6 illustrates the protection of the cutaneous environment of the nail,

FIG. 7 illustrates the protection of the digit,

FIG. 8 represents a set of masks for producing the coating on the nail,

FIG. 9 illustrates the possibility of superposing several pieces of film on the nail,

FIG. 10 represents an example of support with which the treatment head can be equipped,

FIG. 11 illustrates the propagation of the heat in the nail, at different distances from the surface, as a function of time,

FIG. 12 represents a variant support,

FIG. 13 represents a so-called SFL emission spectrum of a pulsed polychromatic light emission treatment device,

FIG. 14 represents an example of transmission spectrum of blue goggles suited to an IPL treatment device having the SFL spectrum of FIG. 13,

FIGS. 15a and 15b represent different partial and schematic views of an end-fitting with which the optical head of the device can be equipped.

### DETAILED DESCRIPTION

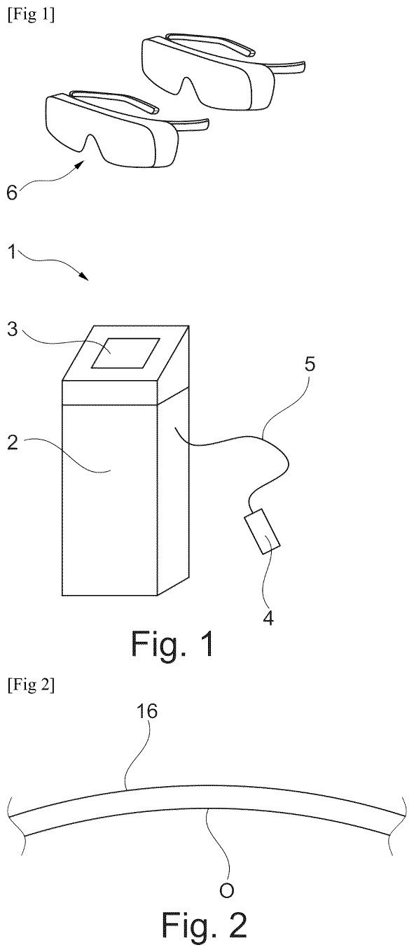

FIG. 1 represents a light pulse emission treatment device 1, comprising a base station 2, provided with a user interface 3, and a treatment head 4 (also called handpiece) linked by a flexible cable 5 to the base station.

The device 1 is for example an IPL-type pulsed light machine, the treatment head 4 comprising at least one flash lamp. The machine can be specific to the treatment of the nails, or parameterizable and have other applications, for example depilation.

Goggles 6 can be used jointly with the machine, in accordance with the teaching of the application EP15738647.5 in the name of the applicant.

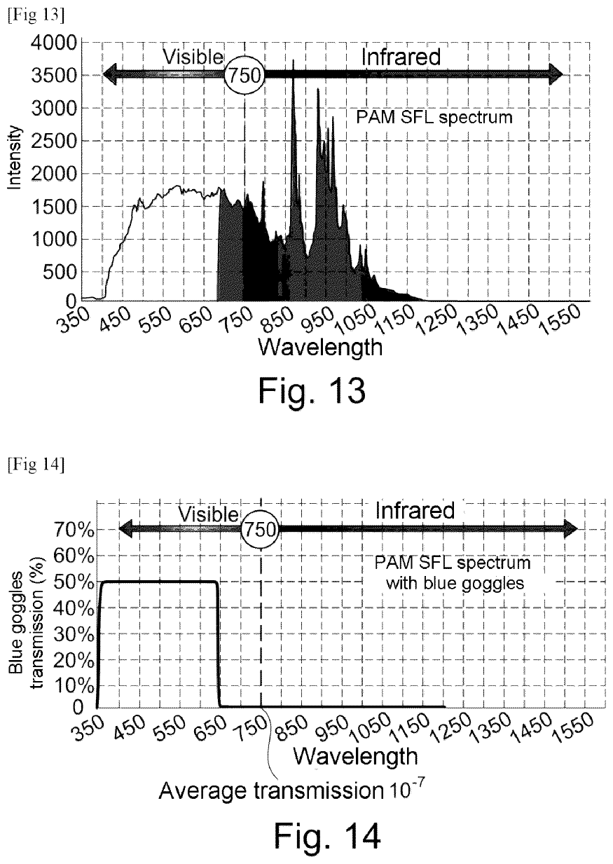

The light emitted can have the spectrum illustrated in FIG. 13, and the goggles 6 can have the spectral transmission factor of FIG. 14.

That allows the operator using the device not to be dazzled by the light emitted, and thus be able to easily control the application of the light during the implementation of the treatment.

The treatment head 4 can contain the flash lamp or lamps, as is the case conventionally for a machine of IPL type.

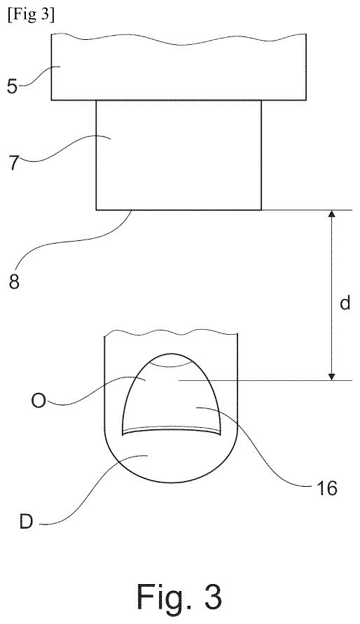

The treatment head 4 has an optical guide 7, one end of which defines a light output window 8, as illustrated in FIG. 3. The nail O to be treated is placed under this output window 8, with a distance d between the nail and the output window for example of between 3 and 6 cm.

In accordance with the invention, a coating 16 that significantly absorbs the light emitted by the output window 8 covers the nail. This coating preferably exhibits a reflectivity less than 50% and/or a transmissivity less than 50%, so as to heat up significantly under the effect of the light flashes.

This coating 16 is preferably composed of a film of an opaque plastic material that is resistant to heat, for example a polyimide (Kapton) or PTFE, of black color. This film can be adhesive or adhere to the nail electrostatically. The transmissivity measured on the Joules-meter is for example 13% for polyimide and 6% for PTFE, for given films of black color, suited to the implementation of the invention.

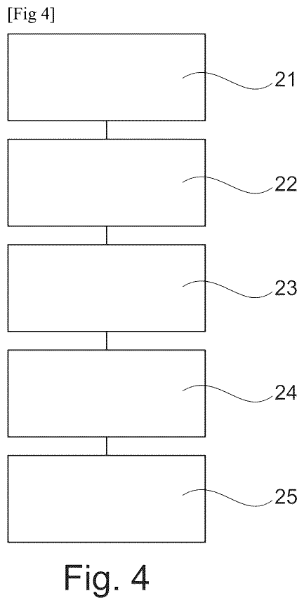

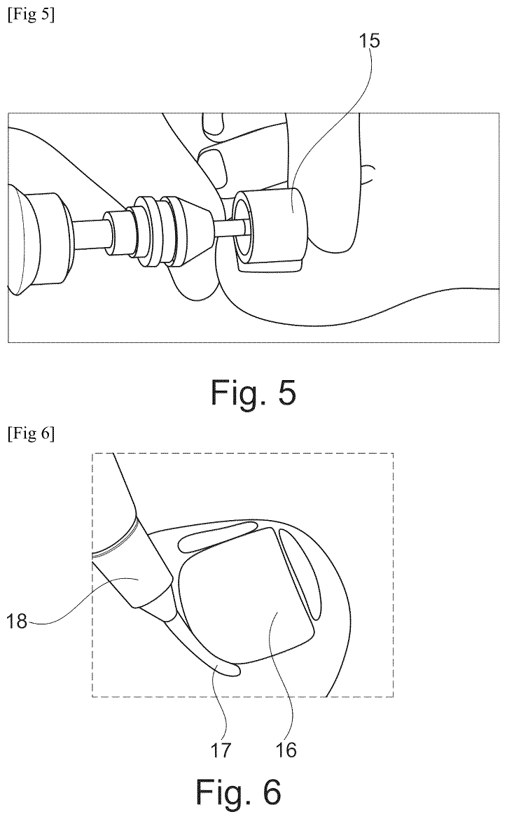

The nail treatment method comprises, as illustrated in FIG. 4, a first step of preparation of the nail to reduce its thickness and smooth its surface. This operation is for example performed using a grinder 15 of a handheld tool, as illustrated in FIG. 5.

Next, in the step 22, the coating 16 is applied to the nail.

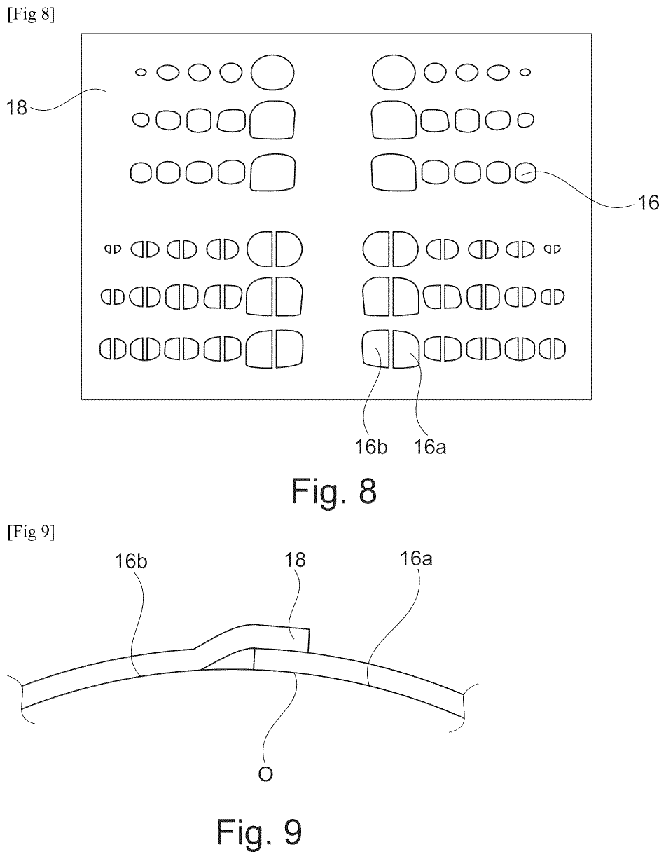

A coating 16 in the form of a patch precut to the form of the nail, supplied on an antiadhesive support 18, as illustrated in FIG. 8, is for example used.

The support 18 can bear patches 16 of different formats, to adapt to the size of the nail. Some patches may correspond to only a part of the surface of the nail, which is the case for example of the patches 16a and 16b. Two patches can be positioned on the nail with an overlap between them, as illustrated in FIG. 9. In this case, it can be begun by positioning a patch starting from one edge of the nail, then the other patch is positioned starting from the opposite edge, and glued onto the first in the overlap zone.

Once the nail O to be treated is provided with the absorbent coating 16, in the step 23, the skin in its region in contact with the nail around the latter can be protected, by depositing a reflective coating 17 on the skin, as illustrated in FIG. 6. This coating 17 is for example a water-dispersible white ink, applied by means of an applicator 18, for example of applicator tip type.

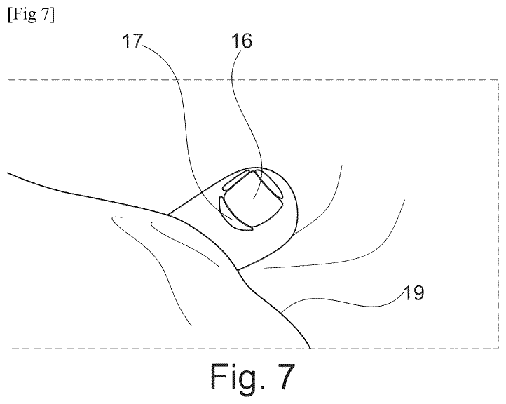

It is also possible to protect a part of the skin of the digit using a white sheet 19 of a flexible material, as illustrated in FIG. 7.

The light treatment takes place in the step 24, and consists in exposing the coating to one or more bursts of flashes in the example considered. At the end of each burst, the user can feel the heat brought to the nail. The step 24 can be repeated as indicated above. During the treatment, the nail is preferably maintained at a predefined distance from the light output window of the treatment head 4, for example using a shim interposed between the digit and the handpiece, or, better, a dedicated support, as detailed hereinbelow.

Next, a last step 25 can consist in removing the coating 16, for example by peeling, as well as the white coating, by washing the digit.

This last step can take place in situ or in the home of the person treated, if appropriate.

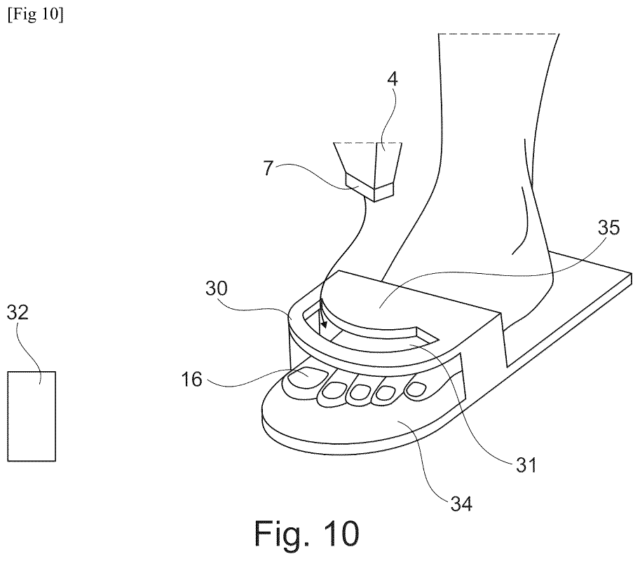

FIG. 10 represents an example of support 30 intended to maintain the light output window at a predefined distance from the nails of the feet. It has a soleplate 34 defining a reception surface for receiving the foot, and a raised part 35 linked to the soleplate 34 and under which the foot is fitted, provided with an aperture 31 positioned above the nail or nails to be treated, for the passage of the light emitted by the head 4 of the device to the nail or nails. The aperture 31 can have a form that makes it possible to engage the optical guide 7 of the head therein, while maintaining the latter at a predefined distance from the soleplate 34. In the example illustrated, the aperture 31 is reniform, and can guide the optical conduit of the head 4 of the device in a scanning movement over the nails of the feet. For example, the operator begins the treatment in proximity to one end of the aperture 31, treats one or two nails, then displaces the conduit in the guide to treat the subsequent nail or nails.

One or more interdigital separators 32 can be used, if necessary, by being disposed between the toes.

The support can, if appropriate, be arranged to be fixed removably onto the treatment head 4, for example by a snap-fitting, guide way or other link, for example by screwing.

As an example, FIG. 12 represents an example of such a support 40. It can be seen that this support 40 can comprise interdigital separators 32.

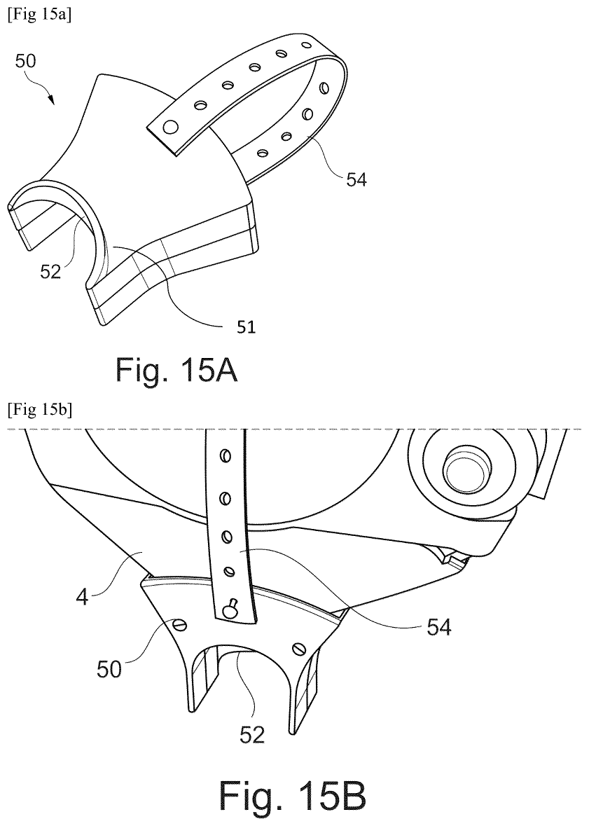

The device according to the invention can also comprise an end-fitting 50 making it possible to guide the light to the nail or nails to be treated, as represented in FIG. 15a and FIG. 15b.

The end-fitting 50 for example comprises a thin strap 54 allowing it to be fixed to the optical head 4 of the device, and a tubular body having a reception zone 51 for the digit or digits for which the nail is to be treated. The reception zone for example accommodates one or two digits, depending on their size.

The reception zone 51 can comprise a concave incurved edge 52 which allows the end-fitting to rest on the digit during the treatment, such that the nail of this digit is positioned to receive the light emitted by the optical head 4. The light is guided from the optical head to the nail through the tubular body, which preferably has a reflective internal surface, white in the example considered.

### Example

A nail affected by a mycosis is prepared by sanding it to reduce the thickness of the nail to 1-2 mm; the sanding can be performed with a nail sander, provided with a single-use abrasive cylinder. The nail can be cleaned with alcohol. Next, a coating is applied consisting of a film of black non-adhesive Kapton onto the nail, and the skin around the nail is protected as described above. The coating can be in the form of two D-shaped patches, disposed so as to overlap on the nail on their rectilinear edges.

The machine used in this example is of IPL type and emits a polychromatic light with the SFL spectrum of FIG. 13. The operator and the person being treated wear blue goggles, the latter almost totally blocking the radiation emitted by the machine by having the spectral transmission factor of FIG. 14.

The nail is subjected to two successive bursts of flashes at a frequency of 3 Hz. Each burst comprises 14 flashes of 4 J/cm² each at the output of the head, which is situated at a distance of 3 cm from the nail. The total duration of each burst is approximately 5 s. The two bursts are spaced apart by approximately 2 mn. The fluence on the coating is approximately four times lower than at the output of the head, i.e. approximately 1 J/cm². The average surface power on the coating is approximately 3 W/cm². The surface temperature of the coating, measured with a thermal camera, exceeds 200° C. for approximately 0.2 s, at the end of the burst.

The photo-thermal effect has three phases. There is, first of all, conversion of the light into heat at the surface of the coating, then transfer thereof into the interior of the tissues, and finally a biological and cellular reaction. The coefficient of spectral absorption of the irradiated surface, associated with the emission spectrum of the source, determines the percentage of photons absorbed and converted into heat. The transfer of the heat is performed by thermal conduction.

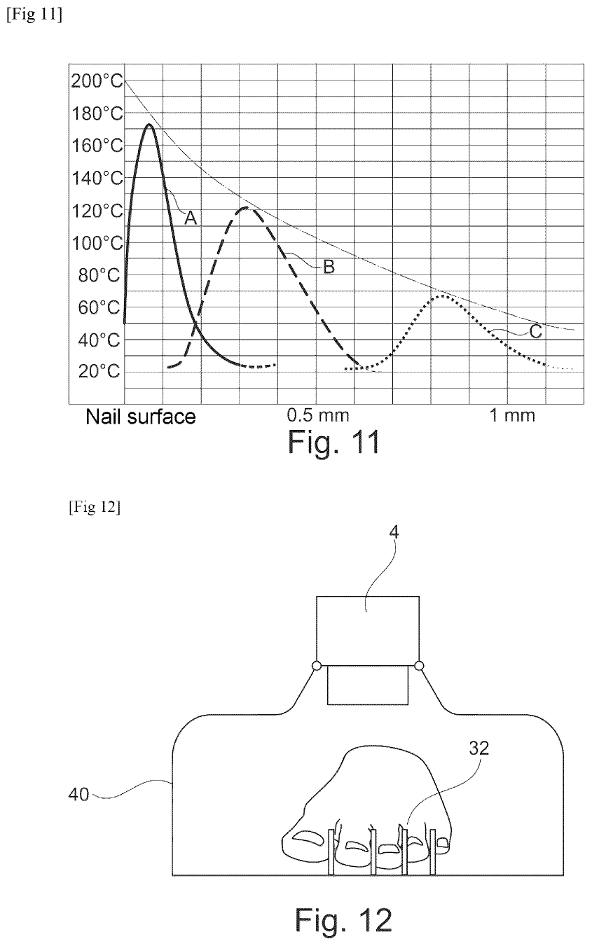

The heat is propagated from the surface of the coating according to a wave, as FIG. 11 illustrates. In this figure, the temperature is represented as a function of time, for different depths from the surface of the nail.

It can be seen that, at the surface of the nail, on contact with the coating, the temperature increases very quickly after the flashes to reach almost 180° C., then decreases because of the diffusion of the heat in the material of the nail (curve A). With increasing distance from the surface of the nail, the curve becomes wider and the maximum temperature decreases (curve B). At the interface between the nail and the underlying tissue (curve C), the maximum temperature is approximately 70° C., which is insufficient to cause a burn given the duration for which this temperature is reached, but sufficient to destroy the germs responsible for the mycosis, located in the thickness of the nail and under the nail.

The invention, with the preparation of the nails that it involves, makes it possible to considerably improve the treatments of the nails by light, by making it possible to work within hitherto unachieved temperature/time pairing zones, notably higher temperatures for shorter times.

However, the invention also makes it possible to treat the nails with maximum surface temperatures of the coating that are lower and for longer times, the drawback of longer treatment being offset by the possibility of using a treatment device having lower light power and fluence.

Obviously, the invention is not limited to the use of a flash lamp IPL machine and it is also possible to use a laser or any other suitable source as light source.

It is also possible to use other types of coatings absorbing the light emitted during the treatment, for example applied in the form of a heat-resistant opaque varnish.

1. A system for treating nails, comprising:
a pulse light emission device making it possible to expose at least one nail to be treated to at least one light pulse,
at least one coating, or an applicator of such a coating to be applied to the nail or nails, absorbing the light emitted by the device and the surface temperature of which can exceed 60° C. under the effect of the light emitted by the device.

2. The system as claimed in claim 1, the coating being of transmissivity less than or equal to 20%.

3. The system as claimed in claim 1, the coating comprising a film of dark color.

4-8. (canceled)

9. The system as claimed in claim 1, the pulsed light emission device being configured to emit a burst of light pulses at a frequency lying between 0.5 and 20 Hz, each pulse exhibiting a fluence such that, given the distance separating an output window of the coating device during use, it lies between 0.5 and 5 J/cm² on the coating surface.

10. The system as claimed in claim 1, the device emitting substantially between the wavelengths of 675 nm and 1200 nm, the system comprising a pair of protective goggles or a protective mask for the operator, comprising a filter absorbing the light emitted by the emission device having a transmission factor less than or equal to 1% above approximately 650 nm.

11. The system as claimed in claim 1, comprising a support for maintaining an output window, through which the light leaves the device, at a predefined distance from a reception surface of the nail to be treated.

12. The system as claimed in claim 11, the support comprising a soleplate defining the reception surface, and a raised part under which the foot can be fitted, provided with at least one aperture under which at least one of the nails can be disposed an optical head being disposed in or above the aperture.

13. The system as claimed in claim 1, comprising a tubular end-fitting having an internal surface that is at least partially reflective, the end-fitting being fixed at one of its ends to the output window of an optical head of the device and comprising, at its other end, a zone for receiving at least one digit such that the nail of this digit is positioned to receive the light emitted by the optical head.

14-33. (canceled)

34. A method for improving the appearance of at least one nail, comprising the step of applying to the nail a coating absorbing the light emitted during the treatment, of positioning an output window of a light pulse emission device relative to the nail such that the light emitted by the device is directed toward the nail, and of subjecting the duly coated nail to at least one pulse so that the temperature measured at the surface of the coating can reach at least 60° C. for a duration of at least 0.1 s.

35. The method as claimed in claim 34, the coating having a reflectivity less than 50%.

36. The method as claimed in claim 34, the coating having a transmissivity less than 50%.

37. The method as claimed in claim 34, the coating resisting a temperature of at least 60° C.

38. The method as claimed in claim 34, wherein a reflecting coating is applied to the periphery of the nail.

39. The method as claimed in claim 34, the absorbent coating being composed of a preformed film.

40. The method as claimed in claim 39, the film being precut substantially to the format of the nail to be treated, or of a part of the nail to be treated.

41. The method as claimed in claim 34, comprising the step of abrading the surface of the nail to be treated prior to the application of the absorbent coating.

42. The method as claimed in claim 34, comprising the step of using a support such that the coating is situated at a predefined distance between 1 and 6 cm from the light output window.

43. The method as claimed in claim 34, wherein a protective screen is disposed on top of the skin of the digit.

44. The method as claimed in claim 34, wherein the nail is subjected to a number of pulses between 2 and 20,

45. The method as claimed in claim 44, wherein after the emission of a burst, the nail is left to cool for a duration of at least 30 s.
